(19) United States
(12) Patent Application Publication
(10) Pub. No.: US 2007/0167529 A1
(43) Pub. Date: Jul. 19, 2007

(54) Antimicrobial compositions for treating fabrics and surfaces

(76) Inventors: Rebecca A. Walton, Newport Beach, CA (US); Bill H. Chapman, Martinsburg, WV (US)

Correspondence Address: HARNESS, DICKEY & PIERCE, P.L.C., P.O. BOX 8910, RESTON, VA, 20195, US

(21) Appl. No.: 11/654,009

(22) Filed: Jan. 17, 2007

Related U.S. Application Data
(60) Provisional application No. 60/759,049, filed on Jan. 17, 2006.

(51) Int. Cl. A01N 31/14; A61K 31/045; A61K 31/075

(52) U.S. Cl. 514/721; 514/724

(57) ABSTRACT

Disclosed are antimicrobial compositions that can be applied to fabrics and/or surfaces for both immediate and residual antimicrobial action against bacteria and fungi in order to suppress the spread of infectious agents. Also disclosed are compositions that may be introduced during the process of laundering fabrics, typically during a later stages such as a rinse, thereby suppressing or eliminating infectious agents remaining in the fabric and providing residual antimicrobial effects that may persist through the expected use of the fabric. The antimicrobial compositions include aqueous solutions including a mixture of ethanol, isopropanol, triclosan (5-chloro-2-(2,4-dichlorophenoxy)phenol), and a surfactant blend or package, the balance of the composition being water. Example embodiments of the surfactant package may include, for example, a blend of 3-methyl-2,5-furandione, 2,7-dimethyl-1-octanol, 2-butyl-1-octanol, 2-methyl-1-decanol, 1-dodecanol, 2-butyl-1-octanol, 2-ethyl-1-dodecanol, 1-tridecanol, 2-tetradecyloxylethanol, 2-dodecyloxyethanol, diethylene glycol monododecyl ether, hexaethylene glycol monododecyl ether, triethylene glycol monododecyl ether and polyoxyethylene sorbitan monooleate.

### PRIORITY STATEMENT

This non-provisional application claims priority under 35 U.S.C. § 119(e) from U.S. Provisional Application No. 60/759,049, which was filed in the U.S. Patent and Trademark Office on Jan. 17, 2006, the contents of which are herein incorporated, in their entirety, by reference.

### TECHNICAL FIELD

Example embodiments relate to antimicrobial compositions useful in a variety of applications including, for example, laundry rinse additives for antimicrobial treatment of fabric and cleaning solutions for disinfecting porous and hard surfaces.

### BACKGROUND OF THE TECHNOLOGY

Studies have repeatedly demonstrated that infectious agents including bacteria, fungus and viruses can be transferred between individuals through contact with the bodily fluids of an infected individual. Such transferred infectious agents are of particular and increasing concern in both acute and long-term healthcare institutions. Accordingly, caregivers and other individuals that come into contact with infected patients or materials will frequently be the recipient of such transfers and thereby become potential vectors for spreading the infection. These types of transfers are of particular concern to those that may be exposed to antibiotic resistant bacteria, for example, methicillin-resistant Staphylococcus aureus (MRSA), work with surgical patients or others with open wounds, and/or immuno-compromised patients that would be particularly susceptible to acquiring such transferred infections.

Accordingly, healthcare and governmental organizations have urged caregivers and healthcare facilities to improve their infection control practices including, for example, wider use of disposable barrier garments, improved hand hygiene and improved clothing hygiene, thereby reducing the odds that a healthcare worker or contaminated item will transfer an infection to a subsequently treated patient.

Indeed, studies published in the American Journal of Medical Quality provide new evidence for those experts who having been arguing that hospitals could prevent many of the growing number of the hospital-acquired infections that afflict patients nationwide, cost billions of dollars to treat and are responsible for thousands, if not tens of thousands, of deaths annually. Rather than accepting some rate of infections as inevitable and unavoidable, health professionals have been encouraged to promote hand-washing among medical staff, take greater care in donning gowns and other infection-preventing clothing during medical procedures, reduce the number of personnel moving in and out of operating rooms, isolate patients as necessary and use antibiotics more selectively to reduce the number of infections and to reduce the likelihood of creating (or selecting for) additional antibiotic-resistant organisms.

Preventing infections, however, can present a delicate balancing act because simple measures such as increased antibiotic use could actually further promote the evolution of the drug-resistant organisms that are responsible for increasing numbers of infections and that increase the odds of negative outcomes, particularly for vulnerable patients. A Pennsylvania survey conducted in 2004 that covered 168 hospitals and 1.6 million patients found that the average hospital stay was nearly 21 days for those patients with hospital-acquired infections as compared to an average of five days for patients that did not acquire such infections. This variation in the hospitalization times was reflected in the corresponding average hospital charge was $185,260 for those with infections, nearly six times the $31,389 incurred by the other patients and the mortality data with about 12 percent of patients with the hospital-acquired infections dying compared with only 2.3 percent of other patients.

Accordingly, there remains a need for antimicrobial compositions that may be used for disinfecting fabrics, both woven and non-woven, as well as the hard and/or porous surfaces found throughout healthcare facilities. As will be appreciated by those skilled in the art, a number of products are currently marketed as potential solutions for one or more of these tasks. Example embodiments of the composition, however, include treatment solutions that may be applied directly to clothing recently soiled by blood or other body fluids for disinfecting the contaminated area, may be applied to fabrics during laundering operations in order to sanitize the fabrics and provide residual antimicrobial performance, and/or may be applied to contaminated surfaces including, for example, floors, trays, doors and/or cabinets for the purpose of sanitizing the surface(s), thereby suppressing the patient-to-patient and/or patient-to-caregiver infections.

### SUMMARY OF THE EXAMPLE EMBODIMENTS

Antimicrobial compositions according to the example embodiments can be sprayed or otherwise applied to clothing to provide both immediate and residual antimicrobial action against bacteria and viruses in order to suppress the spread of infection through contact with contaminated fabrics including, for example, surgical scrubs, lab coats, towels and sheets. Other compositions according to the example embodiments may be introduced during the laundering process, typically during the later stages such as the final rinse, thereby eliminating infectious agents remaining in the fabric and providing residual antimicrobial effects that may persist through the expected use of the fabric.

Example embodiments of the composition are aqueous solutions including a mixture of ethanol, isopropanol, triclosan (5-chloro-2-(2,4-dichlorophenoxy)phenol), and a surfactant blend or package, the balance of the composition being water. Example embodiments include solutions including a blend of C2-C3 alcohols, triclosan and a surfactant package, for example, 1.0-2.5% ethanol, 10.0-13% isopropanol, 1.5-2.5% triclosan and 10-11.5% surfactant package, the balance being water. Example embodiments of the surfactant package may include, for example, a blend of substituted and unsubstituted furandiones, C2-C14 alcohols and ethylene glycol derivatives. Example embodiments of the surfactant package include compositions including 7.5-9% furandione(s), 55.0-57.5% substituted and unsubstituted C2-C14 alcohols and 34-36% ethylene glycol/C8-C14 alcohol ethers.

Example embodiments of the surfactant package include a mixture of, for example, 3-methyl-2,5-furandione, 2,7-dimethyl-1-octanol, 2-butyl-1-octanol, 2-methyl-1-decanol, 1-dodecanol, 2-butyl-1-octanol, 2-ethyl-1-dodecanol, 1-tridecanol, 2-tetradecyloxylethanol, 2-dodecyloxyethanol, diethylene glycol monododecyl ether, hexaethylene glycol monododecyl ether, and triethylene glycol monododecyl ether. Unless otherwise specifically indicated, all of the reported percentages indicated in the specification and claims are reported as weight percents.

Sufficient quantities of these compositions may be added to rinse water during laundering operations as a masterbatch additives to provide a rinse solution retaining antimicrobial activity sufficient to neutralize substantially all bacterial and fungal contamination remaining in the fabric and provide residual antimicrobial activity for a period of time subsequent to the laundering process including, for example, use and subsequent launderings. Alternatively, these compositions may be applied directly to surfaces and/or fabrics by spraying and/or wiping.

### DETAILED DESCRIPTION OF EXAMPLE EMBODIMENTS

The following description is intended to enable one skilled in the art to make and use the compositions and methods as defined by the following claims, and is provided in the context of certain example embodiments. Various modifications to the disclosed embodiments will be apparent to those skilled in the art, and the general principals discussed below may be applied to other embodiments and applications without departing from the scope and spirit of the disclosure.

Compositions according to the example embodiments can be sprayed or otherwise applied to clothing to provide both immediate and residual antimicrobial action against bacteria and viruses in order to suppress the spread of infection through contact with contaminated fabrics including, for example, surgical scrubs, lab coats, towels and sheets. Other compositions according to the example embodiments may be introduced during the laundering process, for example during a the final rinse, thereby eliminating infectious agents remaining in the fabric and providing residual antimicrobial effects that may persist through the expected use of the fabric.

Example embodiments of the composition are aqueous solutions including a mixture of ethanol, isopropanol, triclosan (5-chloro-2-(2,4-dichlorophenoxy)phenol), and a surfactant blend or package. As formulated, the basic composition includes a combination of actives at concentrations sufficient to achieve an isopropanol:ethanol ratio of about 6-7:1, a triclosan:ethanol ratio of about 1-1.5:1; and an isopropanol:surfactant package ratio of about 1.0-1.2:1. Example embodiments of the composition are aqueous solutions including a mixture of ethanol, isopropanol, triclosan (5-chloro-2-(2,4-dichlorophenoxy)phenol), and a surfactant blend or package, the balance of the composition being water. Example embodiments include solutions including a blend of C2-C3 alcohols, triclosan and a surfactant package, for example, 1.0-2.5% ethanol, 10.0-13% isopropanol, 1.5-2.5% triclosan and 10-11.5% surfactant package, the balance being water and one or more additives as discussed in more detail below.

Example embodiments of the surfactant package may include, for example, a blend of substituted and unsubstituted furandiones, C2-C14 alcohols and ethylene glycol derivatives. Example embodiments of the surfactant package include compositions including 7.5-9% substituted and unsubstituted furandione(s), 55.0-57.5% substituted and unsubstituted C2-C14 alcohols and 34-36% ethylene glycol/C8-C14 alcohol ethers. An example embodiment of such a composition may include, for example, 1.7% ethanol, 11.5% isopropanol, 2.1% triclosan and 10.8% of a surfactant package, the balance of the composition being water.

An example embodiment of the surfactant package may include, for example, a blend of 7.8% 3-methyl-2,5-furandione, 0.7% 2,7-dimethyl-1-octanol, 1.5% 2-methyl-1-decanol, 17.1% 1-dodecanol, 5.0% 2-butyl-1-octanol, 1.6% 2-ethyl-1-dodecanol, 9.1% 1-tridecanol, 4.1% 2-tetradecyloxylethanol, 15.1% 2-dodecyloxyethanol, 15.3% diethylene glycol monododecyl ether, 2.7% hexaethylene glycol monododecyl ether and 15.4% triethylene glycol monododecyl ether.

Triclosan has been in use as an antimicrobial for more than 35 years and has been widely accepted as an antibacterial and antifungal active without having raised any particular concerns regarding side effects. Indeed, triclosan is incorporated in products such as toothpastes, soaps and acne treatment compositions that are approved for direct application to patients' skin. Bacteria commonly found on the human body may generally be classified as being “gram-positive” or “gram-negative” with many types of gram-positive bacteria being commonly found on our skin and sometimes referred to as “resident flora.” As suggested by the term, these gram-positive bacteria live naturally on the skin and, in some instances, actually help protect against other potentially more dangerous organisms.

Some of the gram-negative bacteria, however, are not so benign and can cause various infections and illnesses. Unfortunately, gram-negative bacteria also tend to be rather transient and can contaminate your hands as, for example, change a diaper, handle gym equipment, handle food or come into contact with a sick person. It is believed that low levels of triclosan combat both gram-positive and gram-negative bacteria primarily by interfering with a an enzyme that is crucial to the growth of bacteria while higher concentrations may be sufficient to prevent the bacteria from manufacturing the fatty acids they need to build cell membranes. As a result, the normal function of the bacterial cell is disrupted, thereby preventing the bacteria from multiplying or killing the bacteria outright.

When utilized as a laundry rinse additive, sufficient quantities of one or more compositions consistent with the example embodiments may be added to rinse water during laundering operations as a masterbatch additives to produce an antimicrobial rinse solution. The concentration of the active components from the masterbatch should be set so as to provide initial antimicrobial activity sufficient to neutralize substantially all bacterial and fungal contamination remaining in the fabric and provide some residual antimicrobial activity. Depending on the laundry methods and equipment and the type and severity of the initial contamination, it is anticipated that additive concentrations on the order of 0.1 g to 5 g per kilogram of laundered fabric and, perhaps more typically, no more than about 0.5 g per kilogram, may be sufficient to provide satisfactory initial antimicrobial activity.

The treated fabric will also typically exhibit residual antimicrobial activity for a period of time subsequent to the completion of the laundering process comprising, for example, normal use and at least one subsequent laundering. Depending on the specific application intended for the composition, other conventional additives may be incorporated including, for example, UV protectants; fabric care enzymes, dye-transfer inhibitors, anti-redeposition agents, dye sequestrants, dye, pigment and fabric color fixatives, finish protectants, textile lubricant, textile softening agent, hardness and metal ion sequestrants, crystal growth inhibitors, chlorine and/or active oxygen scavengers or neutralizers, processing agents to modify elastic and viscous phase properties, anti-foaming or frothing agents and pH buffer(s).

Fabric care enzymes include, for example, cellulase enzymes for use in combination with cellulosic (cotton) fibers to suppress pilling and fuzzing of cotton fabrics during the washing process. During laundering and normal use, abrasion and fiber damage incurred by the fabric can result in loose fibers, also referred to as “fuzz,” that can, in turn, become entangled to form “pills.” Cellulase enzymes can also remove or reduce existing pilling and fuzzing resulting from normal wear and thereby restore a more “original” fabric appearance during subsequent laundering operations. Further, because damaged fibers are more likely to suffer accelerated dye and/or pigment loss the cellulase enzymes can suppress or delay the appearance of fading.

As will be appreciated by those skilled in the art, the performance of fabric care enzymes may be sensitive to process parameters including, for example, pH (with the performance of cellulase enzymes, for example, tending to improve at lower pH values) and temperature. Other conditions including, for example, the use of wetting agents, other actives and the specific enzyme(s) utilized will also typically affect the performance of the fabric care enzymes to some degree and are routinely taken into account by those skilled in the art when developing a fabric treatment process.

Other fabric care enzymes can include, for example, hydrolases, such as carbohydrases (amylases), proteases and esterases (lipases). As will be appreciated by those skilled in the art, proteases are useful for addressing protein-based stains such as blood and grass stains while amylases are useful for addressing carbohydrate-based and starch-based stains. In most conventional applications it is anticipated that fabric care enzyme concentrations on the order of about 0.2 to about 1% will provide satisfactory performance.

In addition to fabric care enzymes, example embodiments of the compositions may include one or more UV protectants suitable for retarding the fabric degrading effects associated with UVA and/or UVB radiation. Such compounds include free radical scavengers that may be used for light stabilizing and may be used in combination for reducing damage to fabric dyes and finishes that are particularly susceptible to light damage. Other compounds including, for example, conventional fluorescence whitening agents (FWA) that act by absorbing UV light and emitting blue fluorescent light to provide a color brightening effect. FWAs or other optical brighteners are typically used as replacements for older blueing agents previously utilizing for brightening the light yellow coloration associated with laundered cotton fabrics.

FWAs are typically organic compounds which convert UV light (for example, light having a wavelength of 240 to 700 nm) into visible blue light that is perceived as whitening of the treated fabric. FWAs are may generally be classified into one of four major types including cotton FWAs, chlorine resistant FWAs, polyamide FWAs, and polyester FWAs that are, in turn, typically based on one of five principal compounds including stilbene, biphenyl stilbene, coumarin, quinolone, biphenyl pyrazoline and a combination of benzoxazole/benzimidazole with an appropriate conjugated system. It will be appreciated by those skilled in the art that not all UV absorbing compounds will also function as an FWA.

Other light protective materials that may be incorporated into example embodiments of the antimicrobial compositions include free-radical scavengers and/or hindered amine light stabilizers (HALS), both of which are intended to suppress damage from free-radicals generated as a result of irradiation. These actives may also suppress oxidation damage to redox sensitive dyes, fabric and finishes. In most conventional applications it is anticipated that UV protectant concentrations on the order of about 0.05 to about 1% will provide satisfactory performance.

As noted above, a blended surfactant package comprises a substantial portion of the example embodiments of the antimicrobial compositions. Surfactants encompass surface active dispersing, emulsifying and/or solubilizing agents and may generally be classified as anionic, nonionic or cationic surfactants but also includes amphoteric surfactants, zwitterionic surfactants and/or hydrotropes. Nonionic surfactants include, for example, modified polysiloxanes, alkoxylated alcohols, alkoxylated phenol ethers and glycosides. Other surfactants, for example, trialkyl amine oxides may be referred to as “semi-polar” non-ionic surfactants, may also be incorporated. Other nonionic surfactants include C6-C16 linear ethoxylated alcohols (typically averaging about 2 to 20 moles of ethylene oxide per mole of alcohol), C6-C16 linear and branched, primary and secondary ethoxylated and propoxylated alcohols (typically averaging no more than 10 moles of ethylene oxide and less than 10 moles of propylene oxide per mole of alcohol); C8-C16 linear and branched alkylphenoxy (polyethoxy) alcohols (typically averaging 1.5 to 30 moles of ethylene oxide per mole of alcohol) and mixtures thereof.

Surfactants and surfactant blends may also be characterized with regard to their relative solubility between an organic phase, typically octanol, and an aqueous phase. This characteristic ratio may also be referred to as the hydrophilic/lypophilic balance (“HLB”) as determined by surfactant partitioning the organic and aqueous phases. Accordingly, surfactants characterized by higher HLB values are more likely to be more water soluble than surfactants characterized by lower HLB values. Surfactants may also be characterized with respect to a “cloud point” that is defined as the temperature at which a 1% solution of the surfactant turns cloudy upon heating. It is believed that the observed “clouding” is associated with the surfactant coming out of solution as a result of temperature induced dehydration of the ethyloxylate portion of the molecule. Accordingly, surfactants characterized by lower cloud points are generally considered to be less soluble surfactants relative to those surfactants that exhibit improved resistance to dehydration and the associated reduction in solubility.

As will be appreciated by those skilled in the art, compositions according to the example embodiments will typically utilize a blended surfactant package that may include, for example, both nonionic surfactants characterized by a lower range of HLB values that will generally be more capable of solubilizing hydrophobic materials, for example, fragrances and other organics, and nonionic surfactants characterized by higher ELB values, for example, between 5 and 40, that will generally be more capable of coupling the materials into water. Other nonionic surfactants may prove suitable for inclusion in the surfactant package include polyoxyethylene carboxylic acid esters, fatty acid glycerol esters, fatty acid and ethoxylated fatty acid alkanolamides, block copolymers of propylene oxide and ethylene oxide, and block polymers or propylene oxide and ethylene oxide in associated with propoxylated ethylene diamine. In addition, semi-polar nonionic surfactants including, for example, amine oxides, phosphine oxides, sulfoxides and their ethoxylated derivatives may be used, typically sparingly.

As will be appreciated by those skilled in the art, anionic surfactants may include a negatively charged water solubilizing group. Examples of anionic surfactants that would be expected to be suitable for inclusion in the antimicrobial composition include ammonium, substituted ammonium (s, mono-, di-, and triethanolammonium), alkali metals and alkaline earth metal salts of C6-C20 fatty acids and rosin acids, linear and branched alkyl benzene sulfonates, alkyl sulfates, alkyl ether sulfates, alkane sulfonates, alpha olefin sulfonates, hydroxyalkane sulfonates, fatty acid monoglyceride sulfates, alkyl glyceryl ether sulfates, acyl sarcosinates and acyl N-methyltaurides.

Amphoteric and zwitterionic surfactants including an anionic water-solubilizing group, a cationic group or a hydrophobic organic group include, for example, amino carboxylic acids and their salts, amino dicarboxylic acids and their salts, alkyl-betaines, alkyl aminopropylbetaines, sulfobetaines, alkyl imidazolinium derivatives, certain quaternary ammonium compounds, certain quaternary phosphonium compounds and certain tertiary sulfonium compounds.

As noted above, supplemental actives may include dye and pigment anti-redeposition materials, dye-transfer inhibitors and/or dye sequestrants including, for example, polyvinylpyrrolidone (PVP), capable of binding to free dyes released during washing to prevent the undesirable redeposition of the free dyes onto other fabrics present in the solution, thereby suppressing the likelihood of obtaining the proverbial pink undergarments. Dye transfer inhibitors (“DTI”) include solubilized or dispersed substances which act to prevent the discoloration of items by extraneous or free flowing dyes present in the wash solution after having been released from other fabrics being laundered. DTIs may achieve this purpose using a variety of techniques including, for example, suspending the dye in the wash water, solubilizing the dye in a manner unsuitable for redeposition onto another wash item, reducing the affinity of the dye for a textile substrate, fixing the dye to the fabric, trapping the dye and precipitating the dye out of solution. Alternately, DTIs may also adsorb, absorb, or otherwise consume extraneous dyes present in the wash solution in a manner similar to that of a dye absorber. As used herein, the alternate terms “take-up,” “eliminate,” “scavenge” and “sequester” should be understood as being generally equivalent terms for characterizing the mechanism(s) by which DTIs suppress undesirable bleeding and/or color redeposition of extraneous dye or dyes in the wash solution from taking place.

Materials that would generally be expected to perform acceptably as DTIs include, for example, polyvinyl pyrrolidone (PVP), polyvinyl alcohol (PVA), polyvinyl imidazole (PVI), polyamine-N-oxides such as polyvinylpyridine-N-oxide (PVNO), hydrophobicly or cationicly modified PVP, copolymers thereof, cationic starches, minerals including, for example, magnesium aluminate and hydrotalcite, proteins and hydrolyzed proteins, polyethylene imines, polyvinyl oxazolidone, enzymatic systems including peroxidases and oxidases, oxidants, cationic and amphoteric surfactants, as well as propylene oxide reaction products, polyamino acids such as polyaspartic acid or polyhistidine, block co-polymers of ethylene oxide and propylene oxide, polyamines and polyamides, cationic starches, methyl cellulose, carboxyalkyl celluloses such as carboxymethyl and carboxyethyl cellulose, guar gum and natural gums, alginic acid, polycarboxylic acids, cyclodextrins and other inclusion compounds, and mixtures thereof.

PVP for example, is a highly polar nonionic polymer, which also complexes with anionic dyes in aqueous solution. The classes of anionic dyes most commonly used for fabric dying include “direct,”, “reactive,”, and “acid.” The interaction between PVP and dyes in the wash water, however, tends to reduce the amount of dye that is transferred onto clothing. Overuse of dye transfers can, however, cause clothing to lose its brightness and can even change the hue. In extreme cases, dye transfer can cause areas of severe dye staining on clothing. The dyes most readily complexed by PVP seem to include dyes having larger ratios of sulfate (SO³⁻) groups to the molecule size. This type of complexed structure most commonly occurs when using direct dying processes and materials.

Other compounds useful for soil and clay removal and as an anti-redeposition agent are mixtures of polyethylene glycol having a selected weight average molecular weight range of between about 1,000 and about 50,000, more preferably between about 5,000 and about 20,000, and a polyacrylate having a selected weight average molecular weight range of between about 1,000 and about 20,000, more preferably between about 3,000 and about 8,000. Example embodiments of the composition may contain from about 1% to about 20% of a polyethylene glycol/polyacrylate mixture.

Also suitable for consideration for use in compositions according to example embodiments of the composition are lubricating/softening agents that include, for example, silicon-based textile lubricants and textile softening agents that tend to bind or coat textiles and thereby reduce inter-fiber friction and fiber surface friction. These components will typically reduce fabric abrasion during both machine agitation and during wear and include, for example, silicon oils, siloxanes, silicones, siloxanes, polysilicones, polysiloxanes, aromatic silicon compounds, silanes and derivatives thereof.

The cationic fabric softener compounds that may be useful in the antimicrobial compositions according to the example embodiments include, for example, quaternary ammonium or imidazolinium compounds having at least one quaternary nitrogen atom in the molecule. The quaternary ammonium compounds are characterized by independently selected long chain saturated or unsaturated aliphatic hydrocarbon groups each with from C14-C26, halides, for example, chlorides and bromides, nitrates, sulfates, methylsulfates and ethylsulfates. The long chain aliphatic carbon groups can be linear or branched and derived from fatty acids or fatty amines.

Other optional compositions for inclusion in the antimicrobial compositions include, for example, materials for modifying the elastic and viscous phase properties of the compositions. These include thickening agents and viscosity modifying additives suitable for modifying the composition to improve the pouring and handling characteristics, particularly during a dispensing operation. These actives may also contribute to improved product stability, resistance to phase separation and settling of dispersed materials in the composition through elastic modification of the composition phase properties. Included in this category are adjuncts, exemplified by soluble ionic salts, organic salts and hydrotropes that aid the viscosity modifying additive(s) by controlling the ionic strength of the solution. Examples include naturally derived biopolymers such as starch, xantham gum, gum Arabica, derivatized biopolymers such as methyl- and ethyl-cellulose and synthetic polymers such as polyvinyl alcohol. Other thickeners that may be useful in compositions according to the example embodiments include organic, nonionic, water soluble and water swellable polymer including, for example, polyethoxylated urethanes and cellulose ethers such as hydroxyethyl cellulose, methylcellulose, and hydroxypropylmethyl cellulose.

As will be appreciated by those skilled in the art, antimicrobial compositions according to the example embodiments may incorporate a polymeric thickening agent and/or a polymeric mixture capable of suspending relatively large particles while remaining relatively pourable. Specifically, the polymer or mixture are selected to form a continuous, interlocking network system. It is has been established that polymers that require at least some ionic species to be present as a prerequisite for gel formation are generally more susceptible to destabilization by surfactant whether formed as a continuous network or a non-continuous network of gel “bits.”

In general, the polymer or polymer mixture forming the modified viscosity or continuous network system in compositions according to the example embodiments will be of natural origin, specifically one or more polysaccharides. However, it is also possible that the polymer, or one or more polymers in a mixture of polymers, might be a chemically modified natural polymer such as a polysaccharide which has been chemically altered to incorporate and/or modify substituent groups. Polymer compositions including both a synthetic polymer and a natural polymer may also be utilized. In many instances, however, the polymer(s) used will include a natural polysaccharide chain and may be selected from various commercial gums that may, in turn, be characterized as being sourced from a marine plant, a terrestrial plant, microbial polysaccharides and/or polysaccharide derivatives. In addition, gums may be derived from animal sources (e.g., from skin and/or bones of animals) such as gelatin.

Examples of nonionic plant gums include agar, alginates, carrageenan and furcellaran. Examples of terrestrial plant gums include guar gum, gum arabic, gum tragacanth, karaya gum, locust bean gum and pectin. Examples of microbial polysaccharides include dextran, gellan gum, rhamsan gum, welan gum, xanthan gum. Examples of polysaccharide derivatives include carboxymethylcellulose, methyl hydroxypropyl cellulose, hydroxypropyl cellulose hydroxyethyl cellulose, propylene glycol alginate, hydroxypropyl guar and modified starches. It is anticipated that suspending polymer/polymer mixture concentrations of from 0.1 to 0.6% of the total polymer content will generally provide acceptable results. In addition to the gum content, additional thickening agents or structurants including, for example, polysaccharide derivatives such as carboxymethyl cellulose and methylhydroxypropyl cellulose may also be included.

As will be appreciated by those skilled in the art, antimicrobial compositions according to the example embodiments may incorporate polymeric aqueous pH and buffering agents for maintaining acceptable product pH during storage. Such additives are particularly important in combination with fabric care enzymes in order to provide conditions favorable for enzyme stability and/or enzyme activity. These actives can also improve phase stability by retarding or suppressing precipitation and/or separation of other actives that are sensitive to changes in solution pH resulting from absorption of atmospheric gases such as carbon dioxide during storage. Examples of such actives include mono-, di- and tri-ethanolamine and their hydrochloride salts, and ethanolamine derivatives. Mineral acids such as hydrochloric acid, sulfuric acid and nitric acid are examples of suitable pH adjusters along with organic acids such as sulfonic acid, sulfamic acid and citric acid.

Other miscellaneous compounds that may be incorporated into the antimicrobial compositions to improve the aesthetic appeal to the consumer including, for example, dyes and fragrances and/or improve the processing and handling of the compositions including, for example, foaming agents, anti-foaming agents, foam reducing agents, wetting agents depending on the desired product characteristics.

As noted above, example embodiments of the antimicrobial compositions may be utilized in various ways including, for example, as a laundry additive for sanitizing fabrics, particularly those that will be utilized in environments in which infectious contamination is a significant possibility. In these example embodiments, a predetermined volume or amount of the antimicrobial composition may be mixed with laundry solutions (those comprising an aqueous solution of detergents and/or other actives) or water to form a treatment solution that can be used for pretreating and/or presoaking fabrics.

In other example embodiments of methods of use of the antimicrobial composition, one or more of the compositions may be introduced during the washing cycle, in combination with or in sequence with the laundry or fabric detergent composition or other solution. In this mode, the fabric care compositions according to the example embodiments are very simple to use. In the main wash cycle, the compositions may be used either alone or in combination with a regular detergent or laundry additive. In other example embodiments, the antimicrobial composition may be used as a rinse additive and introduced either alone or in combination with a fabric softener during a final stage of the laundry operation.

In other applications, aqueous solutions corresponding to example embodiments of the antimicrobial composition may be applied to a cloth or other suitable carrier (for example, a microfiber mop) that is, in turn, wiped across a surface. In other applications, aqueous solutions corresponding to example embodiments of the antimicrobial composition may be applied directly to the surface, e.g., a floor, and then removed with an appropriate tool, e.g., a mop or a floor polishing machine pad. Conversely, aqueous solutions corresponding to example embodiments of the antimicrobial composition may be mixed with water or other suitable solvents to create a secondary cleaning solution that is, in turn, applied to a surface using a mop or other suitable tool. Regardless of the actual means used to apply the antimicrobial compositions to the item which is to be cleaned and sanitized, the actives concentration in the applied composition should be sufficient to address any bacterial or fungal contamination present.

An example embodiment of the antimicrobial compositions was prepared in accord with the composition detailed above in paragraphs [0014] and [0016]. This composition was then added to the rinse water during the laundering of a variety of test fabrics according the NAMSA protocols. The tested fabrics included samples of 100% polyester; 65/35 polyester-cotton blend and 100% cotton and each of the fabric types was, in turn, subjected to tests including an antifungal test (according to American Association of Textile Chemists and Colorists (“AATCC”) 30), an antibacterial test (according to AATCC 100) and a streak test (according to AATCC 147). The antimicrobial composition demonstrated its effectiveness on each of the fabric samples and on each of the AATCC tests, providing kill zones of at least 8 mm in each of the tests. In the AATCC 100 test, for example, the treated fabric exhibited a kill zone of approximately 20 mm.

We claim:

1. An aqueous antimicrobial composition comprising:
a blend of C2-C4 alcohols;
triclosan; and
a surfactant package.

2. The aqueous antimicrobial composition according to claim 1, wherein the blend of C2-C4 alcohols comprises:
isopropanol and ethanol in an isopropanol:ethanol ratio of about 6-7:1.

3. The aqueous antimicrobial composition according to claim 2, wherein:
the composition exhibits a triclosan:ethanol ratio of about 1-1.5:1; and an isopropanol:surfactant package ratio of about 1.0-1.2:1.

4. The aqueous antimicrobial composition according to claim 1, comprising:
1.0-2.5% ethanol;
10.0-13% isopropanol;
1.5-2.5% triclosan; and
10-11.5% surfactant package,
the balance of the composition being water.

5. The aqueous antimicrobial composition according to claim 4, comprising:
1.7% ethanol;
11.5% isopropanol;
2.1% triclosan; and
10.8% of the surfactant package, the balance of the composition being water.

6. The aqueous antimicrobial composition according to claim 1, wherein the surfactant package comprises:
substituted and unsubstituted furandiones, the substituents being C2-C4 alkyl groups; and
substituted and unsubstituted C2-C13 alcohols, the substituents being C1-C14 alkyl groups.

7. The aqueous antimicrobial composition according to claim 6, wherein the surfactant package comprises:
7.5-9% substituted and unsubstituted furandiones;
55.0-57.5% substituted and unsubstituted C2-C14 alcohols; and
34-36% ethylene glycol/C8-C14 alcohol ethers.

8. The aqueous antimicrobial composition according to claim 7, wherein the surfactant package comprises:
8.0-8.4% substituted and unsubstituted furandiones;
56.0-57.5% substituted and unsubstituted C2-C14 alcohols; and
34.5-35.5% ethylene glycol/C8-C14 alcohol ethers.

9. The aqueous antimicrobial composition according to claim 8, wherein the surfactant package comprises:
7.8% 3-methyl-2,5-furandione;
0.7% 2,7-dimethyl-1-octanol;
1.5% 2-methyl-1-decanol;
17.1% 1-dodecanol;
5.0% 2-butyl-1-octanol;
1.6% 2-ethyl-1-dodecanol;
9.1% 1-tridecanol;
4.1% 2-tetradecyloxylethanol;
15.1% 2-dodecyloxyethanol;
15.3% diethylene glycol monododecyl ether;
2.7% hexaethylene glycol monododecyl ether; and
15.4% triethylene glycol monododecyl ether.

10. A method of treating fabric comprising:
forming an aqueous solution comprising no more than about 5% of an antimicrobial composition according to claim 1 based on a total fabric weight;
permeating the fabric with the aqueous solution; and
removing volatile components of the aqueous solution from the fabric to obtain a treated fabric.

11. A method of treating a surface according to claim 10, wherein the antimicrobial composition further comprises:
isopropanol and ethanol in an isopropanol:ethanol ratio of about 6-7:1;
a triclosan:ethanol ratio of about 1-1.5:1; and
an isopropanol:surfactant package ratio of about 1.0-1.2:1.

12. A method of treating a surface according to claim 10, wherein the antimicrobial composition further comprises:
1.0-2.5% ethanol;
10.0-13% isopropanol;
1.5-2.5% triclosan; and
10-11.5% surfactant package, the balance being water.

13. A method of treating a surface according to claim 12, wherein the antimicrobial composition further comprises:
1.7% ethanol;
11.5% isopropanol;
2.1% triclosan; and
10.8% of the surfactant package, the balance of the composition being water.

14. A method of treating a surface according to claim 10, wherein the surfactant package further comprises:
substituted and unsubstituted furandiones, the substituents being C2-C4 alkyl groups; and
substituted and unsubstituted C2-C13 alcohols, the substituents being C1-C14 alkyl groups.

15. A method of treating a surface according to claim 14, wherein the surfactant package further comprises:
7.5-9% substituted and unsubstituted furandiones;
55.0-57.5% substituted and unsubstituted C2-C14 alcohols; and
34-36% ethylene glycol/C8-C14 alcohol ethers.

16. A method of treating a surface according to claim 15, wherein the surfactant package further comprises:
8.0-8.4% substituted and unsubstituted furandiones;
56.0-57.5% substituted and unsubstituted C2-C14 alcohols; and
34.5-35.5% ethylene glycol/C8-C14 alcohol ethers, based on a total surfactant package.

17. The aqueous antimicrobial composition according to claim 16, wherein the surfactant package comprises:
7.8% 3-methyl-2,5-furandione;
0.7% 2,7-dimethyl-1-octanol;
1.5% 2-methyl-1-decanol;
17.1% 1-dodecanol;
5.0% 2-butyl-1-octanol;
1.6% 2-ethyl-1-dodecanol;
9.1% 1-tridecanol;
4.1% 2-tetradecyloxylethanol;
15.1% 2-dodecyloxyethanol;
15.3% diethylene glycol monododecyl ether;
2.7% hexaethylene glycol monododecyl ether; and
15.4% triethylene glycol monododecyl ether.

18. A method of treating a surface comprising:
applying a volume of an antimicrobial composition according to claim 1 to the surface; and
removing volatile components of the antimicrobial composition to obtain a treated surface.

19. A method of treating a surface according to claim 18, wherein the antimicrobial composition further comprises:
1.0-2.5% ethanol;
10.0-13% isopropanol;
1.5-2.5% triclosan; and
10-11.5% surfactant package, the balance being water.

20. A method of treating a surface according to claim 19, wherein the surfactant package includes:
8.0-8.4% substituted and unsubstituted furandiones;
56.0-57.5% substituted and unsubstituted C2-C14 alcohols; and
34.5-35.5% ethylene glycol/C8-C14 alcohol ethers, based on a total surfactant package.
